(19) United States
(12) Patent Application Publication
(10) Pub. No.: US 2012/0103332 A1
(43) Pub. Date: May 3, 2012

(54) Powder Delivery Devices and Methods of Using the Same

(75) Inventor: John D. Parsons, Norfolk (GB)

(73) Assignee: MEADWESTVACO CALMAR GMBH, Hemer (DE)

(21) Appl. No.: 13/382,941

(22) PCT Filed: Jul. 12, 2010
(86) PCT No.: PCT/US10/41643

§ 371 Date: Jan. 9, 2012

Related U.S. Application Data
(60) Provisional application No. 61/225,127, filed on Jul. 13, 2009.

(51) Int. Cl. A61M 15/08; A61M 16/08; A61M 16/10

(52) U.S. Cl. 128/203.15

(57) ABSTRACT

A powder delivery device (100) including a piercing device (180) capable of piercing a blister (102) may be used to deliver powder or product from a blister (102) or other container to a user through a nasal adapter (110) and the powder delivery device (100) may be re-used or refilled as desired.

### CROSS-REFERENCE TO RELATED APPLICATIONS

This application claims the benefit of, and priority to, U.S. Provisional Application No. 61/225,127, entitled “POWDER DELIVERY DEVICES AND METHODS OF USING THE SAME,” filed Jul. 13, 2009, and incorporates the same herein by reference in its entirety.

### BACKGROUND OF THE INVENTION

1. Field of the Invention

The present invention relates to delivery devices capable of delivering a powder and more particularly, to delivery devices capable of delivering a defined dosage of powder.

2. State of the Art

Various types and configurations of nasal delivery devices exist in the market. Many of these devices are configured to deliver a liquid dose of a medicine or other product. While powdered medicines and products are available, there are limited, if any, delivery devices for reliably delivering such products. Further, disposable and resuable devices are not available.

It is therefore desirous to provide a delivery device capable of delivering a powder product or medicament to a user. It is desirable to allow a user to administer such a dose through a nasal delivery device. It is also desirable to have a delivery device that may be refilled, reused, or disposed of.

### BRIEF SUMMARY OF THE INVENTION

According to certain embodiments of the invention, a powder delivery device may include a body, a nasal adapter, a piercing device removably connected to the nasal adapter, an actuator, a bellows, and a spring. One or more vent holes or air passages through the body may provide communication between an interior of the bellows and an exterior of the body. The bellows may be actuated by movement of the actuator to transmit air through the one or more vent holes. The spring may provide precompression for actuation of the bellows and may provide a return mechanism for returning the actuator to a resting position after actuation. In some embodiments, when the bellows is actuated, air from the bellows may pass through the body and into an open blister containing a powder, mixing with the powder and providing a fluid and/or gas source for delivering the powder through the nasal adapter. In some embodiments of the invention, a powder and air mixture may pass through an interior channel of a piercing device which is in communication with an interior portion of the blister and an exterior of nasal adapter. The piercing device may also be used to break or otherwise open the blister.

According to some embodiments of the invention, a piercing device may include a piercer body having a piercer located at one end of the piercing device and one or more piercer outlets located at the other end of the piercing device. The piercer may include one or more piercer inlets open to an interior portion of the piercer body. The one or more piercer outlets may be in communication with the one or more piercer inlets through an interior portion or channel of the piercer body. A piercer may also include one or more airflow guides. A piercer may also include one or more piercing ramps.

According to various embodiments of the invention, a piercing device may be removably attached to the nasal adapter such that it can be removed and replaced after use. In use, the piercing device may pierce a blister placed in a powder delivery device. For instance, when a blister is placed on a body of a powder delivery device and the nasal adapter and piercing device are connected to or attached to the body, such as by twisting, the piercing device may pierce a portion of the blister. At least a portion of the piercer inlet may be positioned in the blister when the nasal adapter is attached or connected to the body such that when the bellows is actuated and air flows from the bellows, into the blister, it picks up the powder and carries it into the piercer inlet, through a channel or passageway in the piercer, and out a piercer outlet.

According to various embodiments of the invention, the powder delivery devices and piercing devices may take on various forms, shapes, and configurations.

### BRIEF DESCRIPTION OF THE DRAWINGS

While the specification concludes with claims particularly pointing out and distinctly claiming particular embodiments of the present invention, various embodiments of the invention can be more readily understood and appreciated by one of ordinary skill in the art from the following descriptions of various embodiments of the invention when read in conjunction with the accompanying drawings in which:

FIG. 1 illustrates a powder delivery device according to embodiments of the invention;

FIG. 2 illustrates a cross-section view of the powder delivery device illustrated in FIG. 1;

FIG. 3 illustrates a blown-apart view of various components of a powder delivery device according to embodiments of the invention;

FIG. 4 illustrates a powder delivery device according to embodiments of the invention;

FIG. 5 illustrates a cross-section view of the powder delivery device illustrated in FIG. 4;

FIG. 6 illustrates a blown-apart view of various components of a powder delivery device according to embodiments of the invention;

FIG. 7 illustrates a powder delivery device according to embodiments of the invention;

FIG. 8 illustrates a cross-section view of the powder delivery device illustrated in FIG. 7;

FIG. 9 illustrates a blown-apart view of various components of a powder delivery device according to embodiments of the invention;

FIG. 10 illustrates a cross-section view of a powder delivery device according to embodiments of the invention;

FIG. 11 illustrates a piercing device for a powder delivery device according to embodiments of the invention;

FIG. 12 illustrates a piercing device for a powder delivery device according to embodiments of the invention;

FIG. 13 illustrates a piercing device for a powder delivery device according to embodiments of the invention;

FIG. 14 illustrates a piercing device for a powder delivery device according to embodiments of the invention; and

FIG. 15 illustrates a piercing device and blister combination according to embodiments of the invention.

### DETAILED DESCRIPTION OF THE INVENTION

According to embodiments of the invention, a piercing device may be used to pierce a blister or a container and to communicate a medicament from within the blister or container to a user. The piercing device may be configured to produce or improve air flow from an exterior of the blister or container through the blister or container such that product in the blister or container may be at least partially removed therefrom. In various embodiments of the invention, a piercing device may be incorporated with a delivery device. The combination of the piercing device and delivery device may allow a user to select and deliver a medication, powder, liquid, or other product from a blister or container using the piercing device.

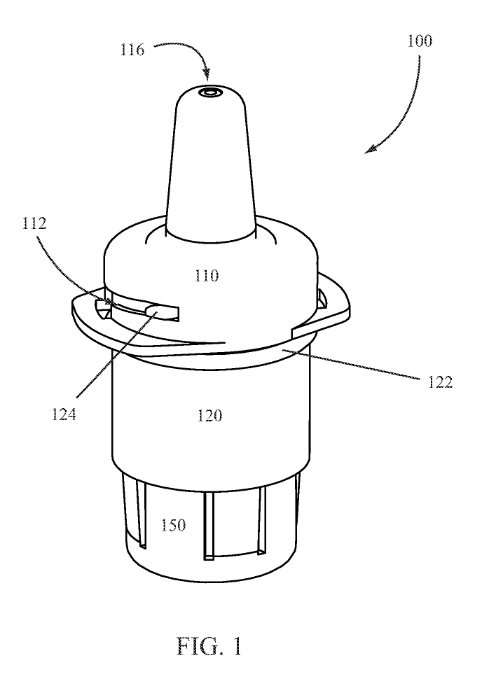

A powder delivery device 100 according to certain embodiments of the invention is illustrated in FIGS. 1 through 3. As illustrated, a powder delivery device 100 may include a nasal adapter 110, a body 120, and an actuator 150. The nasal adapter 110 may be fixed to, or removeably attached to, the body 120. For example, the nasal adapter 110 may include one or more lug guides 112 which may correspond with one or more body lugs 124 on the body 120. One or more body lugs 124 may fit in one or more lug guides 112 allowing the nasal adapter 110 to be twisted about the body 120 such that a bayonet type closure exists between the nasal adapter 110 and the body 120. In other embodiments of the invention, the connection between a nasal adapter 110 and body 120 may include a twist on threaded closure, a snap closure, or any other closure system as desired.

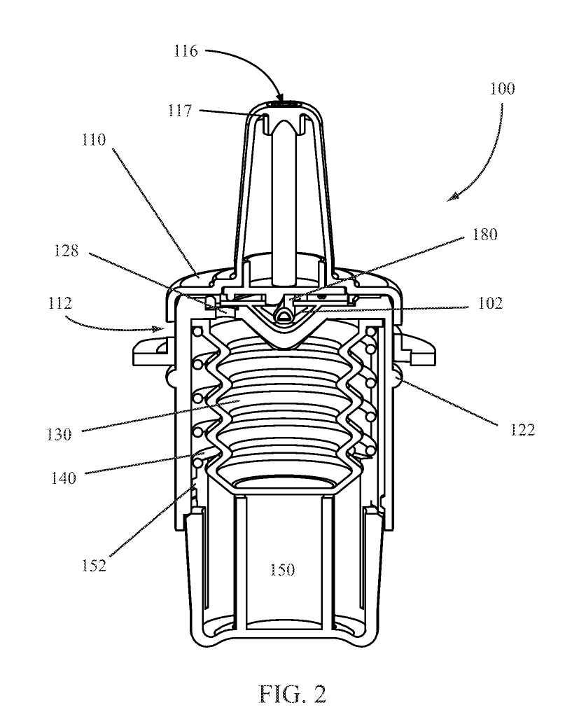

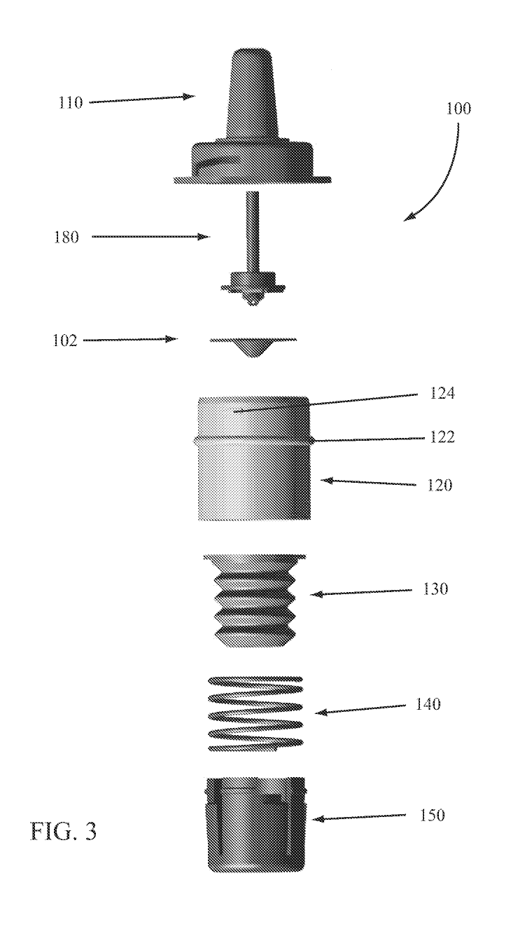

A delivery device 100 may also include a piercing device 180, a blister 102, a bellows 130, and a spring 140 as illustrated in FIGS. 2 and 3. The piercing device 180 may be inserted into or held by the nasal adapter 110. As the nasal adapter 110 is attached and twisted onto the body 120, the piercing device 180 may pierce a blister 102 held in the powder delivery device 100.

According to certain embodiments of the invention, a blister 102 may be added to or removed from a powder delivery device 100 as required. For instance, a user desiring to deliver a dose of medicament contained in a blister 102 may use a powder delivery device 100 according to embodiments of the invention to accomplish such delivery. To operate the powder delivery device 100, the nasal adapter 110 may be removed from the body 120. Removal of the nasal adapter 110 may also remove the piercing device 180 attached to the nasal adapter 110. A blister 102 may be positioned on a support surface of the body 120 and the nasal adapter 110 and piercing device 180 reattached to the body 120. As the nasal adapter 110 is reattached to the body 120, twisting motion of the nasal adapter 110 turns the piercing device 180 which may come into communication with the blister 102. The turning motion of the piercing device 180 may pierce the blister 102 and insert a piercer inlet 183 within the blister 102. The piercing device 180 may also open holes in the blister 102 for allowing air to enter the blister 102 when the powder delivery device 100 is actuated.

A nasal adapter 110 of a powder delivery device 100 according to embodiments of the invention may include one or more openings 116. The one or more openings 116 may be in communication with the piercing device 180. A piercing device connection 117 in the nasal adapter 110 may ensure that a piercing device 180 is secured within the nasal adapter 110. The nasal adapter 110 may also include wings 114. Wings 114 may be configured to assist in the holding of the powder delivery device 100 during actuation thereof.

A body 120 of a powder delivery device 100 according to certain embodiments of the invention may include a body rim 122. A body rim 122 may be used to assist in the assembly of a powder delivery device 100 or may be used as a stop when a nasal adapter 110 is being assembled to a body 120.

In some embodiments of the invention, a body 120 may include one or more air passages 128 through a top portion of the body 120. The air passages 128 may or may not be partially covered by a blister 102 inserted in the powder delivery device 100. If a portion of a blister 102 does overlap at least a portion of an air passage 128, the overlapping portion may be configured to require that a particular force be applied to move the portion of the blister 102 over the air passage 128 such that air will only pass by the blister 102 and into the blister 102 once a predetermined air pressure is reached. In other embodiments of the invention, a valve or other device may be used to allow a precompression force to build up in a bellows 130 prior to air from the bellows being released into a blister 102 in a powder delivery device 100.

The bellows 130, spring 140 and actuator 150 of the powder delivery device 100 may act to force air through a pierced blister 102 and out through the piercing device 180. Actuation of the actuator 150 translates force to the spring 140 and to the bellows 130. The force needed to overcome the spring 140 may ensure that the force being applied to the bellows 130 is sufficient to distribute air from within the bellows 130 through the one or more air passages 128 in the body 120 and into the blister 102. The spring 140 may also assist in returning the actuator 150 to a starting position after a powder delivery device 100 has been actuated. The actuator 150, spring 140 and bellows 130 may be configured as desired to provide a needed amount of air to distribute product from a blister 102 and through the piercing device 180.

In operation, a powder delivery device 100 such as that illustrated in FIGS. 1 through 3 may deliver a desired dosage of a product or powder from a blister 102 inserted in the device to a user. In order to achieve such delivery, a user may rest a blister 102 on the body 120 and attach a nasal adapter 110, including a piercing device 180, to the body 120. Rotation of the nasal adapter 110 to secure it to the body 120 allows the piercing device 180 to pierce the blister 102. Actuation of the actuator 150 applies pressure to the spring 140 and the bellows 130, forcing air through the one or more air passageways 128 and into the pierced blister 102. Air flowing through the blister 102 picks up the product or powder in the blister 102 and carries the product through the piercing device 180 and out the one or more openings 116 in the nasal adapter 110.

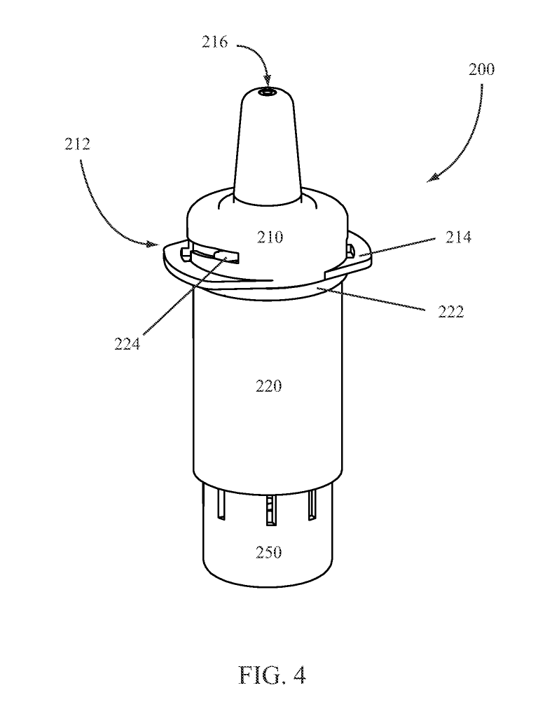

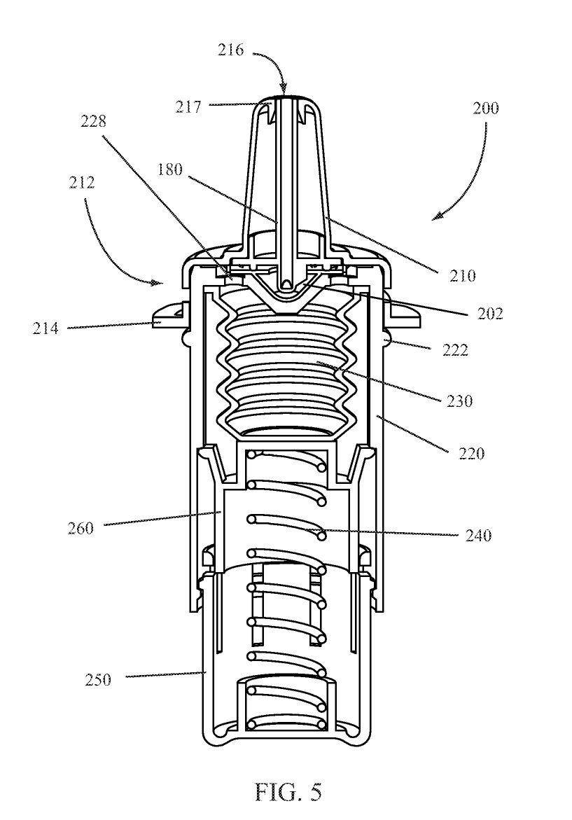

A powder delivery device 200 according to other various embodiments of the invention is illustrated in FIGS. 4 through 6. A powder delivery device 200 may include a nasal adapter 210, a piercing device 180, a body 220, a bellows 230, a flange 260, a spring 240, and an actuator 250. A blister 202 may be situated between the body 220 and a piercing device 180 as desired. The powder delivery device 200 may be assembled as illustrated in FIGS. 5 and 6.

According to various embodiments of the invention, a nasal adapter 210 may include one or more openings 216 in a top portion of the nasal adapter 210. The nasal adapter 210 may also include a piercing device connection 217 which may support a piercing device 180 according to embodiments of the invention. One or more lug guides 212 may also be configured in the nasal adapter 210 for securing the nasal adapter 212 to a body 220 of the powder delivery device 200 by a bayonet-type connection with one or more body lugs 224 on the body 220. In other embodiments of the invention, the body lugs 224 and lug guides 212 may be replaced or substituted with other fastening devices such as threaded closures or snap-type closure systems.

The body 220 of a powder delivery device 200 according to embodiments of the invention may include one or more body rims 222. A body rim 222 may support a nasal adapter 210 on the body 220 or act as a stop for a nasal adapter 210. A body 220 may also include connections to retain a flange 260 and an actuator 250 within the body 220. The body 220 may also be attached to or configured to attach with a bellows 230. One or more air passages 228 within the body 220 may allow air from an interior of the bellows 230 to pass through the wall of the body 220 and into a blister 202 pierced by a piercing device 180.

A powder delivery device 200 such as that illustrated in FIGS. 4 through 6 may include an automatic-fire configuration wherein the powder delivery device 200 delivers a dose of powder from a blister 202 when a predetermined force is applied by a user to the spring 240 of the device. For example, when a user applies a force to the actuator 250 the actuator 250 applies a force to the spring 240. The spring 240 in turn applies a force to the flange 260. The flange 260, however, is set to move once a predetermined force is reached such that until that force is reached, the flange 260 does not activate the bellows 230. Once the predetermined force is reached, the flange 260 fires, acting on the bellows 230 and ejecting air from the bellows 230, through air passages 228 in the body 220 and into blister 202. The air forced through blister 202 picks up the powder in the blister 202 and ejects the powder through the piercing device 180 and out the one or more openings 216 in the nasal adapter 210.

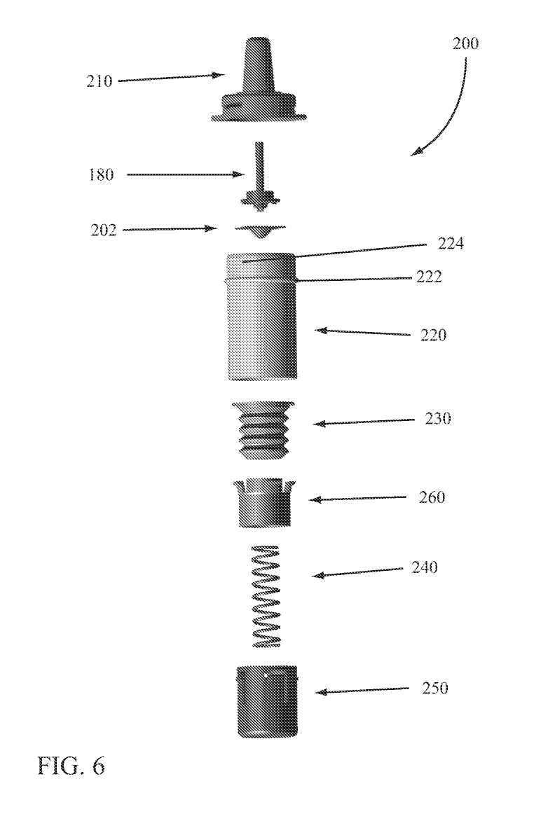

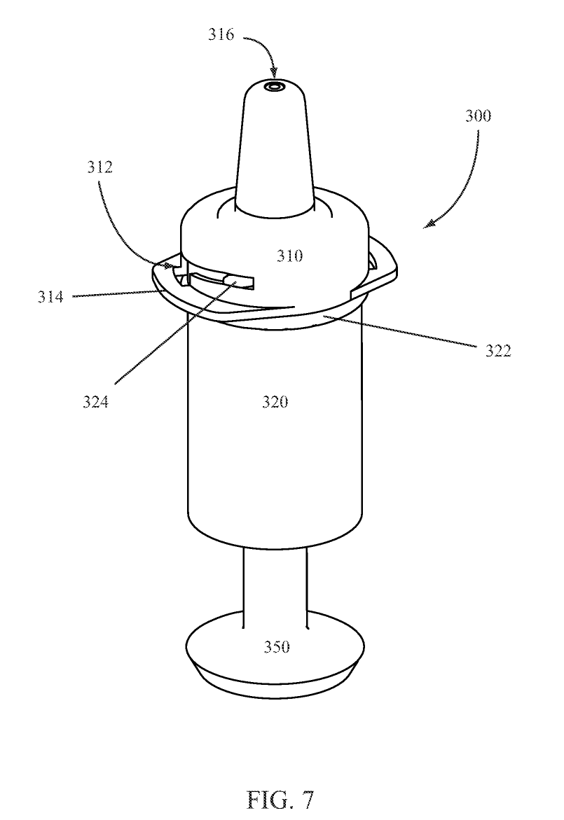

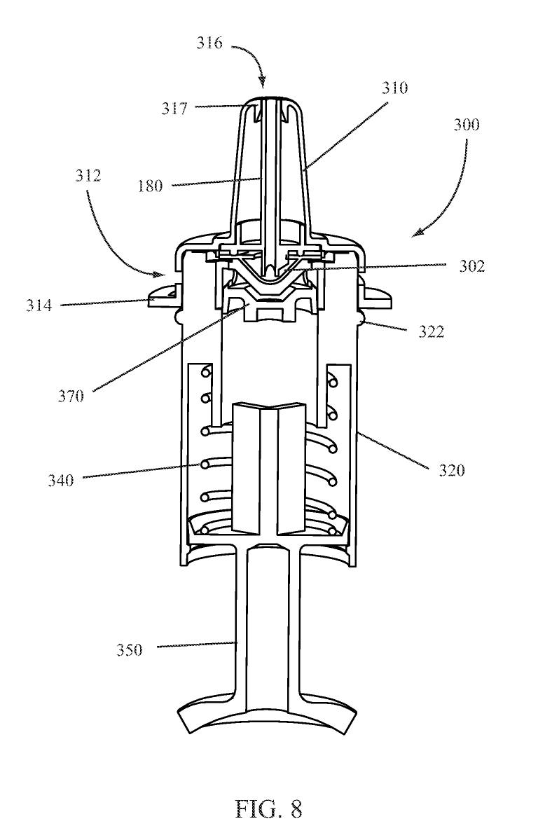

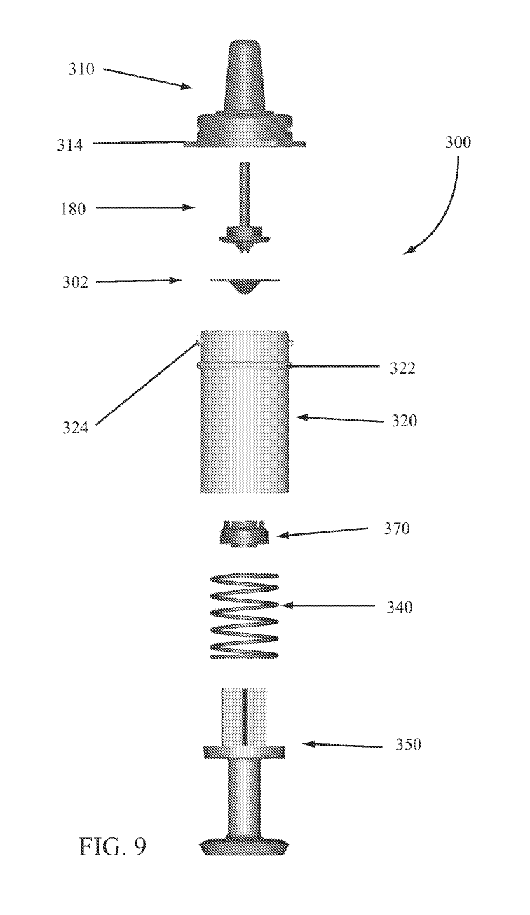

A powder delivery device 300 according to still other embodiments of the invention is illustrated in FIGS. 7 through 9. A powder delivery device 300 may include a nasal adapter 310, a piercing device 180, a body 320, a valve 370, a spring 340, and a piston 350. The powder delivery device 300 may be assembled as illustrated in FIGS. 8 and 9.

According to various embodiments of the invention, a nasal adapter 310 may include one or more openings 316 in a top portion of the nasal adapter 310. The nasal adapter 310 may also include a piercing device connection 317 which may support a piercing device 180 according to embodiments of the invention. One or more lug guides 312 may also be configured in the nasal adapter 310 for securing the nasal adapter 312 to a body 320 of the powder delivery device 300 by a bayonet-type connection with one or more body lugs 324 on the body 320. In other embodiments of the invention, the body lugs 324 and lug guides 312 may be replaced or substituted with other fastening devices such as threaded closures or snap-type closure systems.

The body 320 of a powder delivery device 300 according to embodiments of the invention may include one or more body rims 322. A body rim 322 may support a nasal adapter 310 on the body 320 or act as a stop for a nasal adapter 310. A body 320 may also include connections to retain a valve 370 and a piston 350 within the body 320.

A valve 370 according to various embodiments of the invention may be configured to allow air within the piston chamber formed by the piston 350 and an interior wall of the body 320 to be compressed until a predetermined force or pressure is reached, at which time a valve seal on the valve 370 cracks and allows the air to pass into a blister 302 pierced by the piercing device 380. The valve 370 may include any type of valve 370 suitable for use with a powder delivery device 300 according to embodiments of the invention.

According to embodiments of the invention, a powder delivery device 300 such as that illustrated in FIGS. 7 through 9 may be used to deliver a powder or other product from a blister 302 to a user. For instance, a user may disassemble the nasal adapter 310 from the body 320 and insert a blister 302 into a depression in the body 320. The nasal adapter 310, along with a piercing device 180 housed in the nasal adapter 310, may be connected to or assemble to the body 320. Rotation of the nasal adapter 310 may rotate the piercing device 180 such that the piercing device 180 pierces the blister 302. Depression of the piston 350 compresses spring 340 and air contained in a piston chamber formed between the piston 350 and the body 320. Once the pressure of the air, or the force applied to the air, reaches a sufficient level to crack the valve 370 seal, the valve 370 release the air from the piston chamber into the pierced blister 302. The air picks up the powder or product in the blister 302 and transports it through the piercing device 180 and out the one or more openings 316 in the nasal adapter 310.

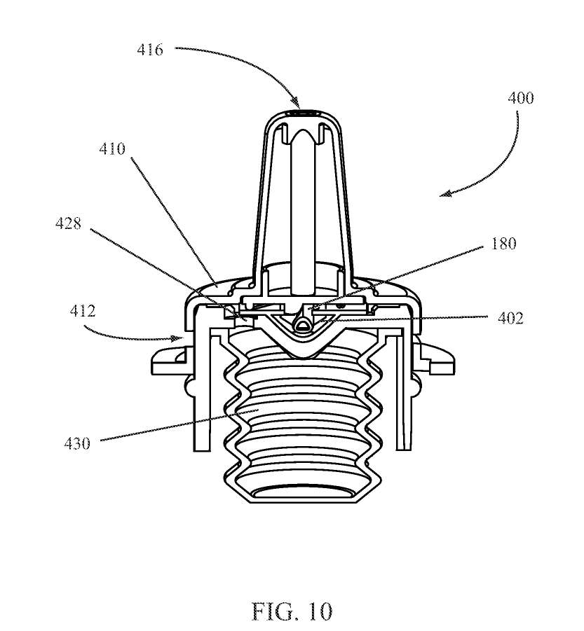

A powder delivery device 400 according to other various embodiments of the invention is illustrated in FIG. 10. As illustrated in FIG. 10, a powder delivery device 400 may include a nasal adapter 410, a body 420, a bellows 430 and a piercing device 180. A blister 402 inserted in between the body 420 and the nasal adapter 410 may be pierced by the piercing device 180 such that powder or a product within the blister 402 may be expelled from the blister 402.

According to embodiments of the invention, the powder delivery device 400 illustrated in FIG. 10 may be used to deliver a dose of powder or product to a user. A user may disassemble the nasal adapter 410 from the body 420. A blister 402 may be placed in a depression in the body 420 and the nasal adapter 410 may be reattached to the body 420. For instance, the nasal adapter 410 may include lug guides 412 which may correspond to body lugs on the body. The lug guides 412 and body lugs may be aligned and the nasal adapter 410 rotated to secure the nasal adapter 410 to the body 420. Other connection mechanisms or systems may also be used. As the nasal adapter 410 is attached to the body 420, a piercing device 180 in the nasal adapter 410 may pierce the blister 402. Actuation of the bellows 430 may force air through the one or more air passages 428 in the body 420 and into the pierced blister 402. Air forced into the blister 402 may carry the powder or product from the blister 402 through the piercing device 180 and out one or more openings 416 in the nasal adapter 410.

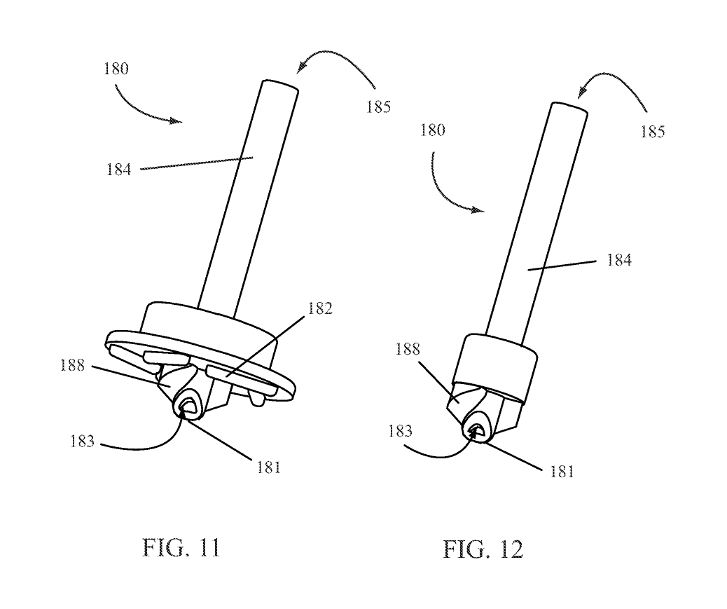

Piercing devices 180 according to various embodiments of the invention are illustrated in FIGS. 11 through 14. According to various embodiments of the invention, a piercing device 180 may include a piercer body 184 having a piercer 181 located at one end of the piercing device 180 and one or more piercer outlets 185 located at the other end of the piercing device 180. The piercer 181 may include one or more piercer inlets 183 open to an interior portion of the piercer body 184. The one or more piercer outlets 185 may be in communication with the one or more piercer inlets 183 through an interior portion or channel of the piercer body 184.

A piercing device 180 according to embodiments of the invention may also include one or more piercing ramps 188. A piercing ramp 188 may include a sharp or defined edge which may tear into a blister 102 as a piercing device 180 is rotated relative to the blister 102. A piercing ramp 188 may also include a defined angle or slope or changing angle or slope configured to tear into a material of a blister 102. For example, a piercing ramp 188 may be configured with an appropriate ramp or slope for tearing into a defined thickness of a particular blister 102 covering material. In other instances, a blister 102 having a thicker or thinner covering material may be paired with a piercing device 180 having piercing ramps 188 configured to tear into the blister 102 and to facilitate delivery of a powder or product through the piercer body 184.

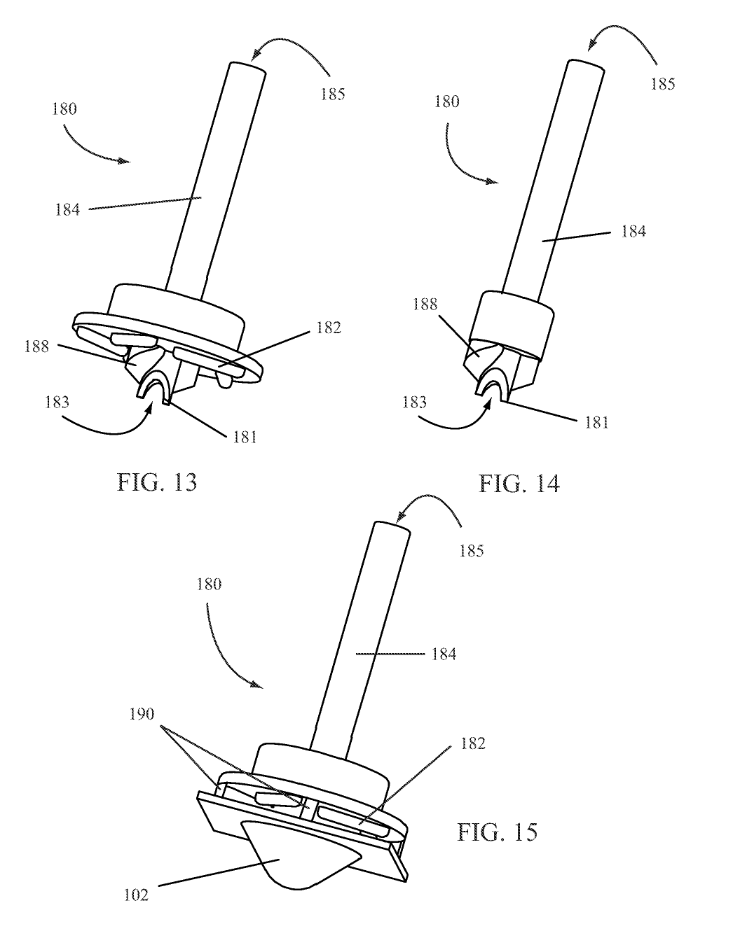

Airflow guides 182 may also be used with a piercing device 180 according to embodiments of the invention. One or more airflow guides 182 configured on a piercing device 180 may direct the flow of air from the powder delivery device into a blister 102 that has been pierced by the piercing device 180. The one or more airflow guides 182 may also be configured or optimized such that a desired airflow is achieved within a pierced blister 102 when a powder delivery device is actuated according to embodiments of the invention. For example, the airflow guides 182 illustrated in FIGS. 11 and 13 may impart a rotational motion or desired turbulence to air passing from within a bellows through an air passage and into a blister 102 associated with the piercing device 180. While certain configurations of airflow guides 182 are illustrated in FIGS. 11 and 13, it is understood that other configurations may also be used with various embodiments of the invention.

A piercer 181 according to embodiments of the invention may include any desired configuration. In certain embodiments of the invention, a piercer 181 may include a single edge such as illustrated in FIGS. 11 and 12. In other embodiments, a piercer 181 may include two or more spikes such as those illustrated in FIGS. 13 and 14. FIGS. 12 and 14 illustrate alternative piercing device 180 configurations according to embodiments of the invention wherein airflow guides 182 are not include as a part of the piercing device 180. In such embodiments, airflow guides 182 may be included on a nasal adapter or other part of a powder delivery device.

According to various embodiments of the invention, a piercing device 180 may be removed from a nasal adapter of a powder delivery device and replaced with a different or new piercing device 180 as desired. For example, once a powder delivery device according to embodiments of the invention is used, powder or other product that has passed through a piercing device 180 may leave a residual amount of powder or other product in the piercing device 180. To prevent cross-contamination of a second dosage of powder or other product delivered by the powder delivery device, a new piercing device 180 may be inserted into the nasal adapter of the powder delivery device. The new piercing device 180 may be used to pierce a new blister 102 inserted into the powder delivery device. In this manner, a powder delivery device may be reused and a piercing device 180 and blister 102 may be used for a single dose.

In still other embodiments of the invention, a blister 102 and a piercing device 180 may be manufactured together or assembled as a unit such that when a user replaces a blister in a powder delivery device they also replace the piercing device 180. For example, a piercing device 180 and blister 102 combination is illustrated in FIG. 15. According to some embodiments of the invention, tabs 190 may connect the blister 102 and the piercing device 180. When the piercing device 180 and blister 102 are seated in a powder delivery device and the nasal adapter is turned or rotated such that the piercing device 180 is rotated relative to the blister 102, the tabs 190 may break, allowing the piercing device 180 to pierce the blister 102 and operate in a desired manner. Other piercing device 180 and blister 102 combinations may also be used with the various embodiments of the invention.

In still other embodiments of the invention, a piercing device and nasal adapter may be manufactured or molded as a single component. For instance, a nasal adapter may include any combination of a piercer, a piercer inlet, piercer ramps, and airflow guides. A nasal adapter and piercing device combination may used as a single use device such that a user may dispose of the nasal adapter and piercing device combination and use a new combination with the body for a second or subsequent delivery of a powder or product from a powder delivery device.

According to other embodiments of the invention, a piercing device 180, such as those illustrated in FIGS. 11 through 15, may be used with a dispenser for delivering other products or fluids. For example, the piercing device 180 may be used with blisters or other containers filled with a fluid product such as a medicine. The piercing device 180 may be used to pierce the blister and to distribute the fluid out of the piercing device 180 delivery opening. A piercing member 180 according to embodiments of the invention may be used with other delivery devices capable of providing or delivering powder, liquid, or powder and liquid combinations.

While five different configurations of piercing devices 180 according to embodiments of the invention are illustrated, it is understood that the shape, configuration, and functionality are not limited by the illustrated embodiments. According to embodiments of the invention, a piercing device 180 may include any device capable of piercing a blister or other powder or liquid container in a sufficient manner to allow the contents of the blister or other container to be at least partially evacuated from the blister or other container through a passageway in communication with the piercing device 180.

The nasal adapters, bodies, actuators piercing devices, and other components of various embodiments of the invention may be made from any desirable material. However, in some embodiments the nasal adapters, body, and actuator of a powder delivery device may be made of a resin material or a plastic material. The resin material or plastic material may be molded into a desired shape and assembled as desired. In addition, a piercing device 180 according to embodiments of the invention may be made of a resin material or a plastic material which is molded into a desired shape with a desired piercer 181, shape, and if used, air flow guides 182.

Having thus described certain particular embodiments of the invention, it is understood that the invention defined by the appended claims is not to be limited by particular details set forth in the above description, as many apparent variations thereof are contemplated. Rather, the invention is limited only be the appended claims, which include within their scope all equivalent devices or methods which operate according to the principles of the invention as described.

1. A powder delivery device, comprising:
a body;
a nasal adapter;
a piercing device between the nasal adapter and the body;
a blister between the piercing device and the body, wherein the blister contains a powder;
a bellows;
a spring; and
an actuator.

2. The powder delivery device of claim 1, further comprising a connection between the body and the nasal adapter.

3. The powder delivery device of claim 2, wherein the connection comprises a closure selected from the group consisting of a bayonet closure, a threaded closure, and a snap closure.

4. The powder delivery device of claim 2, wherein the connection further comprises:
at least one body lug integral with the body; and
at least one lug guide integral with the nasal adapter wherein the at least one body lug fits within the at least one lug guide.

5. The powder delivery device of claim 1, wherein the piercing device comprises:
a piercer body having a first end and a second end;
a piercer located at a first end of the piercer body;
at least one piercer inlet located in the piercer;
at least one piercer outlet located at a second end of the piercer body; and
an interior portion of the piercer body between the at least one piercer inlet and the at least one piercer outlet.

6. The powder delivery device of claim 5, further comprising at least one piercing ramp.

7. The powder delivery device of claim 5, further comprising at least one airflow guides.

8. (canceled)

9. The powder delivery device of claim 1, further comprising a medicament contained within the blister.

10. The powder delivery device of claim 1, further comprising at least one air passage through the body.

11. The powder delivery device of claim 1, wherein the bellows is positioned in an interior of the body, the spring is positioned on an interior of the body and an exterior of the bellows and the actuator is in communication with the spring and the bellows.

12. The powder delivery device of claim 1, further comprising a flange positioned in the body and wherein the bellows is positioned in an interior of the body between the flange and the body, the actuator is positioned between a wall of the flange and an interior wall of the body, and the spring is positioned on an interior of the actuator and flange between the actuator and the flange.

13. A powder delivery device, comprising:
a nasal adapter;
a body;
a piercing device;
a blister containing a powder located between the body and the piercing device;
a valve;
a spring; and
a piston.

14. The powder delivery device of claim 13, wherein the piercing device is positioned between the nasal adapter and the body.

15. The powder delivery device of claim 14, wherein the piercing device further comprises:
a piercer body;
a piercer located at one end of the piercer body;
a piercer outlet located at an opposite end of the piercer body from the piercer; and
at least one piercing ramp.

16. The powder delivery device of claim 14, wherein the piercing device further comprises:
a piercer body;
a piercer located at one end of the piercer body;
at least one piercer inlet located adjacent to the piercer;
a piercer outlet located at an opposite end of the piercer body from the piercer;
at least one air flow guide; and
at least one piercing ramp.

17. A method for delivering a powder, comprising:
providing a blister containing a powder;
providing a powder delivery device comprising:
a body;
a nasal adapter;
a piercing device;
a bellows;
a spring; and
an actuator;
inserting the blister in the powder delivery device between the body and piercing device;
breaking the blister with the piercing device; and
actuating the actuator to dispense the powder.

18. The method of claim 17, wherein inserting the blister in the powder delivery device and breaking the blister further comprises:
removing the nasal adapter from the body of the powder delivery device;
inserting the blister onto the body of the powder delivery device;
twisting the nasal adapter onto the body of the powder delivery device, wherein the twisting rotates the piercing against the blister and breaks the blister.
